(19) United States
(12) Patent Application Publication
(10) Pub. No.: US 2026/0325519 A1
(43) Pub. Date: Oct. 22, 2026

(54) METHOD AND PHARMACEUTICAL COMPOSITION FOR USE IN THE TREATMENT OF FOCAL CORTICAL DYSPLASIA

(71) Applicants: INSTITUT NATIONAL DE LA SANTÉ ET DE LA RECHERCHE MÉDICALE, Paris (FR); UNIVERSITÉ D'AIX MARSEILLE, Marseille (FR); CENTRE NATIONAL DE LA RECHERCHE SCIENTIFIQUE, Paris (FR); UNIVERSITÉ DE BORDEAUX, Bordeaux (FR)

(72) Inventors: Valérie CREPEL, Marseille Cedex 09 (FR); Christophe MULLE, Bordeaux Cedex (FR); Céline BOILEAU, Marseille Cedex 9 (FR); Alfonso REPRESA-BERMEJO, Marseille Cedex 9 (FR); Séverine DEFORGES, Bordeaux Cedex (FR)

(21) Appl. No.: 19/486,199

(22) PCT Filed: May 29, 2024
(86) PCT No.: PCT/EP2024/064839

§ 371 Date: Nov. 20, 2025

(30) Foreign Application Priority Data
May 30, 2023 (EP) 23305851.0

(51) Int. Cl. A61K 31/513; A61P 25/08

(52) U.S. Cl. CPC A61K 31/513; A61P 25/08

(57) ABSTRACT

The present invention relates to the treatment of focal cortical dysplasia. In this study, the inventors showed that GluK2-containing kainate receptors (KARs) play a key role in epileptiform activities in neocortex in an animal model of type II FCD (FCDII) demonstrating that KARs can constitute a major antiepileptic target to treat chronic epilepsy in FCD. Thus, the present invention relates to an inhibitor of GluK2-containing kainate receptors (KAR) for use in the treatment of focal cortical dysplasia (FCD) in a subject in need thereof.

### FIELD OF THE INVENTION

The present invention relates to an inhibitor of GluK2-containing kainate receptors (KARs) for use in the treatment of focal cortical dysplasia (FCD) in a subject in need thereof.

### BACKGROUND OF THE INVENTION

Focal Cortical Dysplasia (FCD) is a cortical developmental malformation responsible for drug-resistant epilepsies in both children and adults representing around 20% of individuals who undergo epilepsy surgery (Lamberink et al., 2020). FCD is characterized by cortical dyslamination (FCDI) and the presence of dysmorphic cytomegalic neurons (FCDIIA) and balloon cells (FCDIIB) (Sisodiya et al. 2009; consensus International League Against Epilepsy 2011: https://www.ilae.org; Blümcke et al. 2011; Najm et al. 2022). During the past decade genetic investigations, particularly of human resected tissue, identified perturbations of the mammalian target of rapamycin (mTOR) signalling pathway as a major cause of FCDII (Baldassari et al 2019). The majority of FCD patients present focal seizures, sometimes as focal infantile spasms characterized by stereo-EEG (SEEG) bursts of rapid rhythms (Baldassari et al 2019).

### SUMMARY OF THE INVENTION

In this study, the inventors showed that GluK2-containing kainate receptors (KARs) play a key role in epileptiform activities in neocortex in an animal model of type II FCD (FCDII) demonstrating that KARs can constitute a major antiepileptic target to treat chronic epilepsy in FCD.

Thus, the present invention relates to an inhibitor of GluK2-containing kainate receptors (KARs) for use in the treatment of focal cortical dysplasia (FCD) in a subject in need thereof. Particularly, the invention is defined by its claims.

### DETAILED DESCRIPTION OF THE INVENTION

A first object of the invention relates to an inhibitor of GluK2-containing kainate receptors (KARs) for use in the treatment of focal cortical dysplasia (FCD) in a subject in need

Particularly, the patient with FCD has an epilepsy.

Thus, the invention relates to an inhibitor of GluK2-containing KARs for use in the treatment of epilepsy in patient with FCD.

In one embodiment, the inhibitor of the invention can prevent either the activation, the expression, or the formation of GluK2, or of the heteromeric complex GluK2/GluK5.

In another embodiment, the inhibitor of the invention can also prevent either the activation, the expression, or the formation of GluK2-containing KARs (i.e heteromeric complexes GluK2/GluK1, GluK2/GluK3 or GluK2/GluK4).

Thus, in a particular embodiment, the invention relates to an inhibitor of the heteromeric complex GluK2/GluK5 for use in the treatment of focal cortical dysplasia (FCD) in a subject in need thereof.

According to the invention, the term “GluK2-containing kainate receptors” denotes all heteromeric receptor containing at least one unit of the subunit GluK2 (including the heteromeric complex GluK2/GluK1, GluK2/GluK3, GluK2/GluK4 or GluK2/GluK5).

As used herein, the IUPHAR-2009-term “GluK2” (also known as GluR6) denotes a subunit of the kainate receptor (KAR) family of ionotropic glutamate receptors and is encoded by the gene grik2. Its Entrez reference number is: 2898 and its Uniprot reference number is: Q13002.

As used herein, the term “GluK2/GluK5 receptor” denotes a heteromeric complex formed by the kainate receptor subunits GluK2 and GluK5 [see for example Reiner et al., 2012, Ruiz et al, 2005].

As used herein, the IUPHAR-2009-term “GluK1”, “GluK2”, GluK3”, “GluK4” or “GluK5” (previously known as KA2) denotes subunits of the kainate receptor family of ionotropic glutamate receptors.

As used herein, the term “inhibitor of GluK2-containing kainate receptors” or “inhibitor of the receptor GluK2” denotes a compound or molecule which prevents the activation of the pathway receptor or a compound or molecule which prevents the formation of the GluK2-containing kainate receptors or a compound or molecule which prevents the synaptic localization of GluK2-containing kainate receptors. Thus, the inhibitor of the invention may be a molecule which binds to the GluK2 KAR subunit receptor to prevent the activation or the formation of the GluK2-containing KARs (including the heteromeric complex GluK2/GluK5). The term “inhibitor of the receptor GluK2” also denotes an inhibitor of the expression of grik2, the gene coding for the protein GluK2.

As used herein the term “Focal Cortical Dysplasia” or “FCD” denotes a chronic neurological condition characterized by chronic and recurrent seizures (epilepsy) which originates in the neocortex. This disease is different from acute seizures in naïve brain tissue since it results from alterations of cortical developmental processes.

In one embodiment, the epilepsy can be classified according to the electroclinical syndromes following the Classification and Terminology of the International League Against Epilepsy (ILAE) [Berg et al., 2010]. These syndromes can be categorized by age at onset, distinctive constellations, including epileptic syndromes that can benefit from surgery, and structural-metabolic causes: (A) age at onset: (i) neonatal period includes Benign familial neonatal epilepsy (BFNE), Early myoclonic encephalopathy (EME), Ohtahara syndrome. (ii) Infancy period includes Epilepsy of infancy with migrating focal seizures, West syndrome, Myoclonic epilepsy in infancy (MEI), Benign infantile epilepsy, Benign familial infantile epilepsy, Dravet syndrome, Myoclonic encephalopathy in nonprogressive disorders. (iii) Childhood period includes Febrile seizures plus (FS+), Panayiotopoulos syndrome, Epilepsy with myoclonic atonic (previously astatic) seizures, Benign epilepsy with centrotemporal spikes (BECTS), Autosomal-dominant nocturnal frontal lobe epilepsy (ADNFLE), Late onset childhood occipital epilepsy (Gastaut type), Epilepsy with myoclonic absences, Lennox-Gastaut syndrome, Epileptic encephalopathy with continuous spike-and-wave during sleep (CSWS), Landau-Kleffner syndrome (LKS), Childhood absence epilepsy (CAE). (iv) Adolescence-Adult period includes Juvenile absence epilepsy (JAE) Juvenile myoclonic epilepsy (JME), Epilepsy with generalized tonic-clonic seizures alone, Progressive myoclonus epilepsies (PME), Autosomal dominant epilepsy with auditory features (ADEAF), Other familial temporal lobe epilepsies. (v) Variable age onset includes Familial focal epilepsy with variable foci (childhood to adult), Reflex epilepsies. (B) Distinctive constellations (epileptic syndromes that can benefit from surgery) include Mesial Temporal Lobe Epilepsy (MTLE), Rasmussen syndrome, Gelastic seizures with hypothalamic hamartoma, Hemiconvulsion-hemiplegia-epilepsy. (C) Epilepsies attributed to and organized by structural-metabolic causes include Malformations of cortical development (Focal cortica dysplasia, Hemimegalencephaly, heterotopias, etc.), Neurocutaneous syndromes (tuberous sclerosis complex, Sturge-Weber, etc.), Tumor, Infection, Trauma, Angioma, Perinatal insults, Stroke, Etc.

In a particular embodiment, the epilepsy may be a benign Rolandic epilepsy, a frontal lobe epilepsy, an infantile spasms, a juvenile myoclonic epilepsy, a juvenile absence epilepsy, a childhood absence epilepsy (pyknolepsy), a hot water epilepsy, a Lennox-Gastaut syndrome, a Landau-Kleffner syndrome, a Dravet syndrome, a progressive myoclonus epilepsies, a reflex epilepsy, a Rasmussen's syndrome, a temporal lobe epilepsy, a limbic epilepsy, a status epilepticus, an abdominal epilepsy, a massive bilateral myoclonus, a catamenial epilepsy, a Jacksonian seizure disorder, a Lafora disease or photosensitive epilepsy.

In one embodiment, the epilepsy is a chronic epilepsy.

In another embodiment, the epilepsy can be a refractory epilepsy.

As used herein, the term “refractory epilepsy” denotes an epilepsy which is refractory to current pharmaceutical treatment, that is to say that current pharmaceutical treatment doesn't allow a treatment of patients (see for example Englot et al., 2014).

In a particular embodiment, the refractory epilepsy is a chronic refractory epilepsy.

As used herein, the terms “treatment” and “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “subject” denotes a mammal, such as a rodent, a feline, a canine, and a primate. Particularly, a subject according to the invention is a human. More particularly, the subject is suffering from FCD or from an epilepsy with FCD.

In one embodiment, the inhibitor according to the invention may be a low molecular weight compound, e.g. a small organic molecule (natural or not). The term “small organic molecule” refers to a molecule (natural or not) of a size comparable to those organic molecules generally used in pharmaceuticals. The term excludes biological macromolecules (e.g., proteins, nucleic acids, etc.). Preferred small organic molecules range in size up to about 10000 Da, more preferably up to 5000 Da, more preferably up to 2000 Da and most preferably up to about 1000 Da.

In a particular embodiment, the inhibitor according to the invention is selected from the group consisting of UBP310, UBP302, UBP316 and NBQX.

In a particular embodiment, the inhibitor according to the invention is UBP310 an antagonist of the heteromeric kainate GluK2/GluK5 receptor [Pinheiro et al., 2013].

In another particular embodiment, the inhibitor according to the invention is (2S,4R)-4-Methylglutamate (SYM2081), a inhibitor that is a potent broad-spectrum agonist of kainate receptors; it binds to homomeric and heteromeric kainate receptors including homomeric GluK1, and GluK2 receptors and heteromeric GluK1/GluK2, GluK1/GluK5 and GluK2/GluK5 receptors [Jane et al., 2009]. This inhibitor is currently used to selectively block kainate receptor signaling via desensitization of the receptors in control and epileptic conditions [Jane et al., 2009] [Li et al., 1999; Cossart et al., 2002; Epsztein et al., 2005; Epsztein et al., 2010; Joseph et al., 2011].

In one embodiment, the inhibitor according to the invention is an antibody. Antibodies directed against GluK2-containing kainate receptors (thus against the subunit GluK2) can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against GluK2-containing kainate receptors can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce anti-GluK2-containing kainate receptors single chain antibodies. Compounds useful in practicing the present invention also include anti-GluK2-containing kainate receptors antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to GluK2-containing kainate receptors.

Humanized anti-GluK2-containing kainate receptors antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Then, for this invention, neutralizing antibodies of GluK2-containing kainate receptors are selected.

In one embodiment, the inhibitor according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by Exponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of GluK2-containing kainate receptors are selected.

In one embodiment, the inhibitor according to the invention is a polypeptide. In a particular embodiment the polypeptide is an antagonist of GluK2-containing kainate receptors and is capable to prevent the function of GluK2-containing kainate receptors and particularly the recruitment or stabilisation of synaptic GluK2/GluK5 receptors or KARs. Particularly, the polypeptide can be a mutated GluK2 subunit protein or a similar protein without the function of GluK2 subunit. The polypeptides of the invention may be produced by any suitable means, as will be apparent to those of skill in the art. In order to produce sufficient amounts of polypeptide or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known. When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli. In specific embodiments, it is contemplated that polypeptides used in the therapeutic methods of the present invention may be modified in order to improve their therapeutic efficacy. Such modification of therapeutic inhibitors may be used to decrease toxicity, increase circulatory time, or modify biodistribution. For example, the toxicity of potentially important therapeutic inhibitors can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications. Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa). In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In another embodiment, the inhibitor GluK2-containing kainate receptors according to the invention is an inhibitor of GluK2 subunit gene expression.

Small inhibitory RNAs (siRNAs) can also function as inhibitors of GluK2 subunit expression for use in the present invention. GluK2 subunit gene expression can be reduced by a small double stranded RNA (dsRNA), or a vector or construct causing the production of a small double stranded RNA, such that GluK2 subunit gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, GJ. (2002); McManus, MT. et al. (2002); Brummelkamp, TR. et al. (2002); U.S. Pat. Nos. 6,573,099 and 6,506,559; and International Patent Publication Nos. WO 01/36646, WO 99/32619, and WO 01/68836).

Ribozymes can also function as inhibitors of GluK2 subunit gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of GluK2 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays. Both antisense oligonucleotides and ribozymes useful as inhibitors of G-CSF gene expression can be prepared by known methods. These include techniques for chemical synthesis such as, e.g., by solid phase phosphoramadite chemical synthesis. Alternatively, antisense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing GluK2. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles are provided in Kriegler, 1990 and in Murry, 1991. Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter can also be, e.g., a viral promoter, such as CMV promoter or any synthetic promoters.

In a particular embodiment, an endonuclease can be used to abolish the expression of the gene, transcript or protein variants of GluK2 subunit.

Indeed, as an alternative to more conventional approaches, such as cDNA overexpression or downregulation by RNA interference, new technologies provide the means to manipulate the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the error prone non homologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8: e2671.), zebrafish (Hwang et al., 2013, PLoS One, Vol. 8: e68708.), C. elegans (Hai et al., 2014 Cell Res. doi: 10.1038/cr.2014.11.), bacteria (Fabre et al., 2014, PLoS Negl. Trop. Dis., Vol. 8: e2671.), plants (Mali et al., 2013, Science, Vol. 339: 823-826), Xenopus tropicalis (Guo et al., 2014, Development, Vol. 141: 707-714.), yeast (DiCarlo et al., 2013, Nucleic Acids Res., Vol. 41: 4336-4343.), Drosophila (Gratz et al., 2014 Genetics, doi: 10.1534/genetics.113.160713), monkeys (Niu et al., 2014, Cell, Vol. 156: 836-843.), rabbits (Yang et al., 2014, J. Mol. Cell Biol., Vol. 6: 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56: 122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpfl which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

Accordingly, one object of the present invention relates to a method of treating a focal cortical dysplasia (FCD) in a subject in need thereof comprising administering to the subject a therapeutically effective amount of an inhibitor of the GluK2-containing KARs.

In order to test the functionality of a putative inhibitor of the GluK2-containing kainate receptors, a test is necessary. For that purpose, both in vitro and/or in vivo systems can be used, such as cell lines, primary neuronal cultures, a recombinant system (Pinheiro et al., 2013), acute or organotypic cortical slices for in vitro systems, and rodent models of FCD as an in vivo system. Depending on the type of inhibitors, the efficacy can be determined using different methods. Here are some examples: (i) measuring the levels of GluK2 subunits via Western Blot or proteomics; (ii) quantifying GluK2 RNA using qPCR; or (iii) assessing the amplitude of the evoked or synaptic currents mediated by GluK2-containing kainate receptors in brain slices, or in HEK293 cells transfected to express recombinant GluK2-containing KARs.

### Therapeutic Composition

Inhibitor of the invention may be administered in the form of a pharmaceutical composition, as defined below.

In another aspect, the invention relates to a therapeutic composition comprising an inhibitor of the GluK2-containing KARs for use in the treatment of focal cortical dysplasia (FCD) in a subject in need thereof.

According to the invention, the inhibitor of the GluK2-containing KARs or the therapeutic composition of the invention are administrated in a therapeutically effective amount.

Any therapeutic agent of the invention may be combined with pharmaceutically acceptable excipients, and optionally sustained-release matrices, such as biodegradable polymers, to form therapeutic compositions.

As used herein, the term “therapeutically effective amount” or “effective amount” refers to an amount effective, at dosages and for periods of time necessary, to achieve a desired therapeutic result. A therapeutically effective amount of the inhibitor or the composition of the present invention may vary according to factors such as the disease state, age, sex, and weight of the individual, and the ability of the inhibitor or the composition of the present invention to elicit a desired response in the individual. A therapeutically effective amount is also one in which any toxic or detrimental effects of the inhibitor or the composition are outweighed by the therapeutically beneficial effects. The efficient dosages and dosage regimens for the inhibitor or the composition of the present invention depend on the disease or condition to be treated and may be determined by the persons skilled in the art. A physician having ordinary skill in the art may readily determine and prescribe the effective amount of the inhibitor or the composition of the invention required. For example, the physician could start doses of the inhibitor or the composition of the present invention employed in the pharmaceutical composition at levels lower than that required in order to achieve the desired therapeutic effect and gradually increase the dosage until the desired effect is achieved. In general, a suitable dose of the inhibitor or the composition of the present invention will be that amount of the inhibitor which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically and for example, the ability of a inhibitor to inhibit cancer may, for example, be evaluated in an animal model system predictive of efficacy in human tumors. Alternatively, this property of a composition may be evaluated by examining the ability of the inhibitor to induce cytotoxicity by in vitro assays known to the skilled practitioner. A therapeutically effective amount of a therapeutic inhibitor may decrease tumor size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of an antibody of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, intracerebral injection (also known as intraparenchymal injection) or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled antibody of the present invention, fragment or mini-antibody derived from the antibody of the present invention. If desired, an effective daily dose of a pharmaceutical composition may be administered as two, three, four, five, six or more sub-doses administered separately at appropriate intervals throughout the day, optionally, in unit dosage forms. In some embodiments, the monoclonal antibodies of the present invention are administered by slow continuous infusion over a long period, such as more than 24 hours, in order to minimize any unwanted side effects. An effective dose of an antibody of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of an antibody of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Administration may be intravenous, intramuscular, intraperitoneal, intratumoral, intracerebral injection (also known as intraparenchymal injection) or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans. If desired, an effective daily dose of a pharmaceutical composition may be administered as two, three, four, five, six or more sub-doses administered separately at appropriate intervals throughout the day, optionally, in unit dosage forms. In some embodiments, the oligomers of the present invention are administered by slow continuous infusion over a long period, such as more than 24 hours, in order to minimize any unwanted side effects. An effective dose of the inhibitor of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of the inhibitor of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

In other words, the quantity of the immune cell or the population of immune cells administered to a subject in need thereof is between 10⁴ to 10⁹ cells per kg. Particularly, the quantity of cells injected is 10⁶ or 10⁷ cells per kg. The immune cell or the population of immune cells of the invention can be administrated is 1, 2, 3, 4 or 5 times to the subject in need thereof.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the subject, etc.

The pharmaceutical compositions of the invention can be formulated for a topical, oral, intranasal, parenteral, intraocular, intravenous, intramuscular, intracerebral (also known as intraparenchymal) or subcutaneous administration and the like.

Particularly, the pharmaceutical compositions contain vehicles which are pharmaceutically acceptable for a formulation capable of being injected. These may be in isotonic, sterile, saline solutions (monosodium or disodium phosphate, sodium, potassium, calcium or magnesium chloride and the like or mixtures of such salts), or dry, especially freeze-dried compositions. In particular, these may be in organic solvent such as DMSO, ethanol which upon addition, depending on the case, of sterilized water or physiological saline permit the constitution of injectable solutions.

In addition, other pharmaceutically acceptable forms include, e.g. tablets or other solids for oral administration; time release capsules; and any other form currently can be used.

In each of the embodiments of the treatment methods described herein, the inhibitorcells of the invention are delivered in a manner consistent with conventional methodologies associated with management of the disease or disorder for which treatment is sought. In accordance with the disclosure herein, an effective amount of the inhibitor cells of the invention administered to a subject in need of such treatment for a time and under conditions sufficient to prevent or treat the disease or disorder.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

In a particular embodiment, the inhibitor or the composition according to the invention may be delivered directly into the brain to directly reach neurons:

Methods of delivery of the inhibitor or the composition of the invention to neurons and/or astrocytes includes generally any method suitable for delivery the inhibitor or the composition of the invention to the neurons such that at least a portion of cells of a selected synaptically connected cell population is transduced. The inhibitor or the composition of the invention may be delivered to neuronal cells of the central nervous system. Generally, the inhibitor or the composition of the invention is preferentially delivered to cortical neurons but without excluding the possibility of delivering the inhibitor or composition of the invention to neurons in other areas of the central nervous system such as neurons in the spinal cord, brainstem (medulla, pons, and midbrain), cerebellum, diencephalon (thalamus, hypothalamus), telencephalon (hippocampus, corpus striatum, cerebral cortex), or combinations thereof, or preferably any suitable subpopulation thereof.

To deliver the inhibitor or the composition of the invention specifically to a particular region and to a particular population of cells of the CNS, the inhibitor or the composition of the invention may be administered by stereotaxic microinjection. For example, patients have the stereotactic frame base fixed in place (screwed into the skull). The brain with stereotactic frame base (MRI compatible with fiducial markings) is imaged using high resolution MRI. The MRI images are then transferred to a computer which runs stereotactic software. A series of coronal, sagittal and axial images are used to determine the target (site of inhibitor or composition injection) and trajectory. The software directly translates the trajectory into 3 dimensional coordinates appropriate for the stereotactic frame. Burr holes are drilled above the entry site and the stereotactic apparatus positioned with the needle implanted at the given depth. The inhibitor or the composition of the invention is then injected at the target sites.

Additional routes of administration may also comprise local application of the inhibitor or the composition of the invention under direct visualization, e.g., superficial cortical application, or other non-stereotactic application. The inhibitor or the composition of the invention may be delivered intrathecally, in the ventricle or by intravenous injection.

The target cells of the inhibitor or the composition of the invention of the present invention are cells of the central nervous systems of a subject afflicted with FCD disease. Preferably the subject is a human being, commonly children and adolescents under 18 years particularly.

However the invention encompasses delivering the inhibitor or the composition of the invention to biological models of the disease. In that case, the biological model may be any mammal at any stage of development at the time of delivery, e.g., embryonic, fetal, infantile, juvenile or adult, preferably it is an adult. Furthermore, the target CNS cells may be essentially from any source, especially nonhuman primates and mammals of the orders Rodenta (mice, rats, rabbit, hamsters), Carnivora (cats, dogs), and Arteriodactyla (cows, pigs, sheep, goats, horses) as well as any other non-human system (e.g. zebrafish model system).

Preferably, the method of the invention comprises intracerebral administration through stereotaxic injections. However, other known delivery methods may also be adapted in accordance with the invention. For example, for a more widespread distribution of the inhibitor or the composition of the invention across the CNS, it may be injected into the cerebrospinal fluid, e.g., by lumbar puncture. To direct the inhibitor or the composition of the invention to the peripheral nervous system, it may be injected into the spinal cord or into the peripheral ganglia, or the flesh (subcutaneously or intramuscularly) of the body part of interest. In certain situations the inhibitor or the composition of the invention can be administered via an intravascular approach. For example, the inhibitor or the composition of the invention can be administered intra-arterially (carotid) in situations where the blood-brain barrier is disturbed or not disturbed. Moreover, for more global delivery, the inhibitor or the composition of the invention can be administered during the “opening” of the blood-brain barrier achieved by infusion of hypertonic solutions including mannitol.

Alternatively, the inhibitor of the invention may be further identified by screening methods as hereinafter described.

Thus, another object of the invention relates to a method for screening an inhibitor of the GluK2-containing kainate receptors.

In particular, the invention provides a method for screening an inhibitor which is an inhibitor of the GluK2-containing kainate receptors for use in the treatment of FCD.

For example, the screening method may measure the binding of a candidate inhibitor of the GluK2-containing kainate receptors, or to cells or membranes bearing the GluK2-containing kainate receptors receptor or a fusion protein thereof by means of a label directly or indirectly associated with the candidate inhibitor. Alternatively, a screening method may involve measuring or, qualitatively or quantitatively, detecting the competition of binding of a candidate inhibitor to the receptor with a labelled competitor (e.g., antagonist).

In a particular embodiment, the screening method of the invention comprises the step consisting of:
a) providing a plurality of cells expressing the GluK2-containing kainate receptors:
b) incubating said cells with a candidate inhibitor;
c) determining whether said candidate inhibitor binds to the GluK2-containing kainate receptors or inhibit the expression of the GluK2 subunit gene of the receptor; and
d) selecting the candidate inhibitor that inhibits the GluK2-containing kainate receptors or that prevents the activation of the pathway receptor or that prevents the formation of the heteromeric complex GluK2/GluK5 for example.

In general, such screening methods involve providing appropriate cells which express the GluK2-containing kainate receptors. In particular, nucleic acids encoding the GluK2 subunit may be employed to co-transfect cells to thereby express the GluK2 receptor. Such a transfection may be achieved by methods well known in the art.

In a particular embodiment, cells may be neuronal cells.

According to a one embodiment of the invention, the candidate inhibitor may be selected from a library of inhibitors previously synthesised, or a library of inhibitors for which the structure is determined in a database, or from a library of inhibitors that have been synthesised de novo or natural inhibitors.

The candidate inhibitor may be selected from the group of (a) proteins or peptides, (b) nucleic acids and (c) organic or chemical inhibitors (natural or not). Illustratively, libraries of pre-selected candidate nucleic acids may be obtained by performing the SELEX method as described in documents U.S. Pat. Nos. 5,475,096 and 5,270,163. Further illustratively, the candidate inhibitor may be selected from the group of antibodies directed against the GluK2 receptor particularly.

### Combination and Kit of Part

In another aspect of the invention, the inhibitor of the invention or the pharmaceutical composition of the invention may comprise a further therapeutic active agent. The present invention also relates to a kit comprising an inhibitor according to the invention and a further therapeutic active agent.

According to the invention, anti-FCD or anti-epileptic agents may be added to the pharmaceutical composition or used in combination with the inhibitor of the invention in the case of the treatment of a FCD.

Anti-FCD or anti-epileptic agents include for example aldehydes, aromatic allylic alcohols like Stiripentol, barbiturates like phenobarbital, methylphenobarbital or barbexaclone, benzodiazepines like clobazam, clonazepam, clorazepate, diazepam, midazolam or lorazepam, bromides like potassium bromide, carbamates, felbamate or cenobamate, carboxamides like carbamazepine, oxcarbazepine or eslicarbazepine acetate, fatty acids like the valproates, vigabatrin, progabide, tiagabine or everolimus.

Another aspect of the present invention relates to i) an inhibitor of the GluK2-containing kainate receptors, and ii) at least one further therapeutic active agent according to the invention, as a combined preparation for simultaneous, separate or sequential use in the treatment of FCD in a subject in need thereof.

As used herein, the term “simultaneous use” denotes the use of an inhibitor of the GluK2-containing kainate receptors and at least one therapeutic active agent occurring at the same time.

As used herein, the term “separate use” denotes the use of an inhibitor of the GluK2-containing kainate receptors and at least one therapeutic active agent not occurring at the same time.

As used herein, the term “sequential use” denotes the use of an inhibitor of the GluK2-containing kainate receptors and at least one therapeutic active agent occurring by following an order.

The invention will be further illustrated by the following figures and examples. However, these examples and figures should not be interpreted in any way as limiting the scope of the present invention.

### FIGURES

FIG. 1: Examples of neocortical neurons immunostained with an antibody against phospho-S6 Ribosomal protein illustrating typical non-cytomegalic (small arrows) and cytomegalic/dysmorphic neurons (big arrows) at lower (top) and higher (bottom) magnifications in an adult rat with Focal Cortical Dysplasia.

FIG. 2: A, Recurrent spontaneous epileptiform discharges recorded ex vivo in an acute neocortical slice with an LFP electrode from an adult FCD rat before (top) and after (bottom) bath application of 5 μM UBP 310 (under hyperexcitable condition: 5 μM gabazine); note the significant reduction (by around 46%) in the number of epileptiform discharges (red stars) in the presence of 5 μM UBP 310 . Inset, example of an epileptiform discharge depicted at an expanded time scale (100 μV/500 ms). B, Bar graph illustrating the average decrease in the frequency of epileptiform discharges in the presence of 5 μM UBP 310 (n=7 slices from 4 FCD rats; Wilcoxon matched-pairs signed rank test, p=0.0156).

FIG. 3: A, Western blotting of GluK2 extracted from a human organotypic cortical slices from a drug-resistant epileptic patient in the presence of an AAV9 control vector or an AAV9 engineered to express a miRNA against grik2; note that GluK2 receptors are highly expressed in human cortical slices (left panel), and that AAV9 with antigrik2 miRNA strongly decrease the GluK2 protein as shown by the bar graph in the right panel. B, Field EPSP (fEPSP) evoked by electrical stimulation (triangle) recorded in a human acute slice from a surgically resected FCD in the absence and in the presence of 5 μM UBP 310; note that fEPSP is strongly reduced in the presence of UBP310. C, Recurrent epileptiform discharges recorded with a local field potential (LFP) electrode in a human acute slice from a surgically resected FCD before and after the application of 5 μM UBP 310 (under hyperexcitable condition: 50 μM 4-AP and 5 μM gabazine); note the strong reduction of the number of epileptiform discharges (red stars) in the presence of UBP310. Inset, example of an epileptiform discharge depicted at an expanded time scale (100 μV/500 ms).

FIG. 4: immunostaining of pS6 in cytomegalic neurons in Focal Cortical Dysplasia. The top left photomicrograph shows typical cytomegalic neurons (examples indicated by arrows) immunostained with an antibody against phosphorylated S6 (pS6) protein in Focal Cortical Dysplasia (FCD, ipsilateral side) in the somatosensory cortex of a Swiss mouse; in the lower photomicrographs, DAPI staining is used to identify nuclei (10× magnification).

FIG. 5: immunostaining of GluK2 in Wild-Type and Focal Cortical Dysplasia Tissues. The top photomicrographs depict the distribution of GluK2 (using the GluK2 Sysy antibody 180003) within cortical layers IV and V of the somatosensory cortex in both wild-type (WT) and FCD Swiss mice; in the lower photomicrographs, DAPI staining is used to identify nuclei (10× magnification). The graph illustrates the immunohistochemical (IHC) analysis of GluK2 expression in cortical layers IV and V, showing increased GluK2 expression in the FCD area compared to WT condition (control, CTL).

### EXAMPLE 1

### Material & Methods

### Ethical Aspects

All experiments conducted on rats are performed in accordance with the European Community Council directives (2010/63/UE) and received approval from the French Ministry for Research, after ethical evaluation by the institutional animal care and use committee of Aix-Marseille University (APAFIS #23362-2019121712305437 v4). For experiments using human resected cerebral tissue, all patients have given their written consent and protocols have been approved by Inserm (N° 2017-00031), AP-HM/CRB-TBM (N° M17-06), and CNRS (Codecoh DC-2020-3863).

### Rat Model of FCD

Our study takes advantage of a rat model of FCD developed in our laboratory (Pelorosso et al 2019) by in utero electroporation of a gain of function mutation of mTOR (MTOR c.6644C>T p.S2215F), that has been found in different FCD patients (Baldassari et al 2019). Rats were housed in cages with ad libitum food/water, controlled temperature (21-23 °C) and 12:12 h light-dark cycle (light period from 8 A.M. to 8 P.M.).

### In Utero Electroporation

Plasmid electroporations were performed with either wild type (WT) or mutant mTOR expression vectors (pCAG-MTOR WT-IRES-GFP or pCAG-MTOR p.S2215F, respectively, 3.0 μg/μL each), or with non-recombinant vector (pCAG-IRES-GFP; 0.5 μg/μL). All constructs were generated by Genscript (Piscataway, NJ) as described before (Pelorosso et al 2019) and were checked by Sanger sequencing.

Timed pregnant Wistar rats (Janvier Labs, Le Genest-Saint-Isle, France) received buprenorphine at 0.03 mg/kg and were anesthetized with sevoflurane (4%) 30 min later. Uterine horns were exposed, and plasmids were microinjected with a PV 820 Pneumatic PicoPump (World Precision Instruments, Sarasota, FL) into the right lateral ventricle of embryonic day 15 embryos (E15), with 0.2% Fast green (F7252, Sigma-Aldrich) dye. Electroporations were performed by delivering 40 V voltage pulses using a BTX ECM 830 electroporator (BTX Harvard Apparatus, Holliston, MA) across tweezer-type electrodes (Nepa Gene Co, Chiba, Japan) pinching the head of each embryo through the uterus.

### Histology and Immunohistochemistry

Brains of adult rats were dissected out after transcardial perfusion with AntigenFix (Diapath) and post-fixed overnight at 4 °C. Brains were sectioned (100 μm) with a vibratome (Leica VT 1000S, Leica, Nussloch, Germany) and processed for immunohistochemistry as free-floating sections. Sections were permeabilized in phosphate-buffered saline (PBS) with 0.3% Triton X-100 for 15 min at room temperature, blocked in PBS with 0.3% Triton X-100 and 10% normal goat serum for 1 h at room temperature and incubated in PBS with 0.3% Triton X-100 and 5% normal goat serum overnight at 4 °C with the following primary antibodies: chicken anti-GFP (1/1000; Thermo Fisher Scientific, Waltham, MA; #A10262) and rabbit anti phospho-S6 Ribosomal protein (1: 200, Cell Signalling Technology, #2215). Sections were washed in PBS and incubated for 2 h at room temperature with the following secondary antibodies: goat anti-chicken Alexa Fluor 488 (1/500; Invitrogen, #A11039), goat anti-rabbit Alexa Fluor 555 (1/500; Invitrogen, #A21428). Sections were counterstained with Hoechst 33342 (1/1000, Thermo Fisher Scientific), washed in PBS and mounted in Fluoromount (Thermo Fisher Scientific). All images were acquired on a LSM800 Zeiss confocal microscope (Zeiss, Jena, Germany) and analyzed with the Fiji software.

Cortical slices from patients with drug-resistant epilepsy Acute and cultured slices at 350 μm thickness were prepared from surgical cortical resections from patient diagnosed with drug-resistant epilepsy (AP-HM, Hôpital de La Timone, Marseille, France; CHU Pellegrin, Bordeaux, France) as previously described (Le Duigou et al., 2018). At DIV 1, a subset of cultured slices were transduced with AAV9 with antigrik2 miRNA applied directly to slices (4E+10 per slice).

Cortical Slices from Rats with FCD

Acute cortical slices of adult rats (60-90 days old) at 350 μm thickness were prepared described (Ackman et al., 2009).

### Electrophysiological Recordings

Prior to recordings, rat or human acute slices were transferred in oxygenated normal cerebrospinal fluid (ACSF) at room temperature (>1 hour). The ACSF contained (in mM, Sigma-Aldrich): 126 NaCl, 3.5 KCl, 1.2NaH2PO4, 26 NaHCO3, 1.3 MgCl2, 2.0 CaCl2 , and 10 glucose (pH 7.4). For electrophysiological recordings, cortical slices were individually transferred to a recording chamber maintained at 30-32 °C and continuously perfused (2-3 ml/min) with oxygenated (95% O2 and 5% CO2) ACSF containing either 5 μM gabazine, or 5 μM gabazine and 50 μM 4-AP in rats and human tissues, respectively. Spontaneous recurrent epileptiform discharges were recorded as local field potentials as previously described (Peret et al., 2014).

### Western Blotting

For western blotting analysis, human cultured cortical slices were snap-frozen in liquid nitrogen and lysed in RIPA buffer (Sigma) added with a protease (Calbiochem) and phosphatase (Pierce) inhibitor cocktails with 3 beads using a high-speed benchtop homogenizer FAST PREP24 (MP Biomedicals). Ten ug of total proteins quantified by Pierce™ BCA protein assay kit (ThermoScientific) were loaded on 4-15% gradient SDS-PAGE gels and transferred to nitrocellulose membranes (Trans-Blot turbo, Bio-Rad). Immunoblotting with human cortical tissue was detected in fluorescent on Li-COR with Image studio software (Biosciences GmbH) using a rabbit anti-GluK2 (Abcam ab124702; 1: 2000), a mouse anti-β actin (Sigma A5316; 1: 5000) in Odyssey blocking buffer TBS (Li-COR) and appropriate 800 nm fluorophore-conjugated secondary antibodies (IRDye 800; 1: 15000). The intensity of the signal for GluK2 was normalized to the β actin signal and thereafter to the control condition.

### Statistical Analyses

All statistical analyses were performed using Prism 6 (Graphpad). Normality of the data distributions was systematically tested using a Shapiro-Wilk test. Comparison of groups was subsequently tested with paired Student's t-tests for normal data sets or with Wilcoxon matched-pairs signed rank test for non-normal data sets. All values are given as mean±SEM. All tests were two-tailed, and the level of significance was set at P<0.05.

### Results

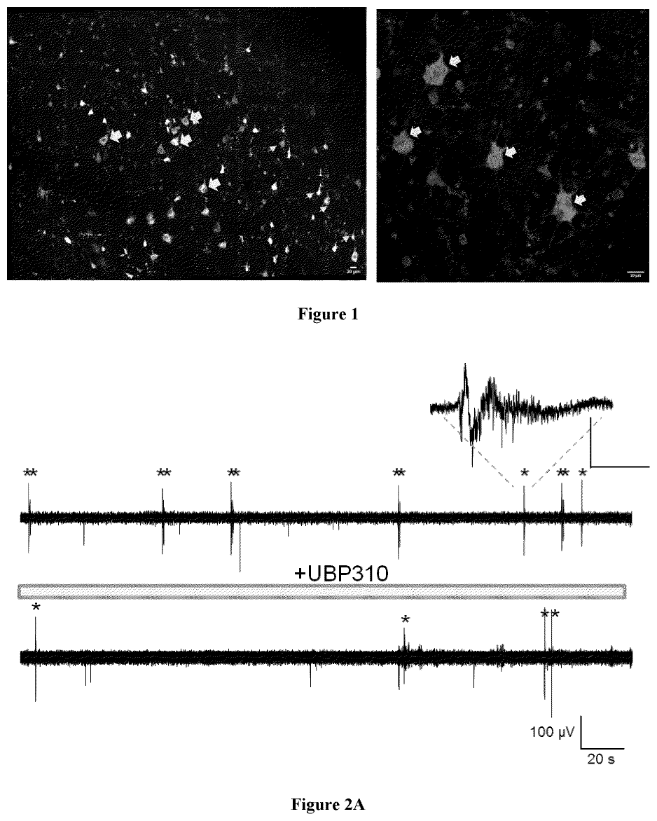

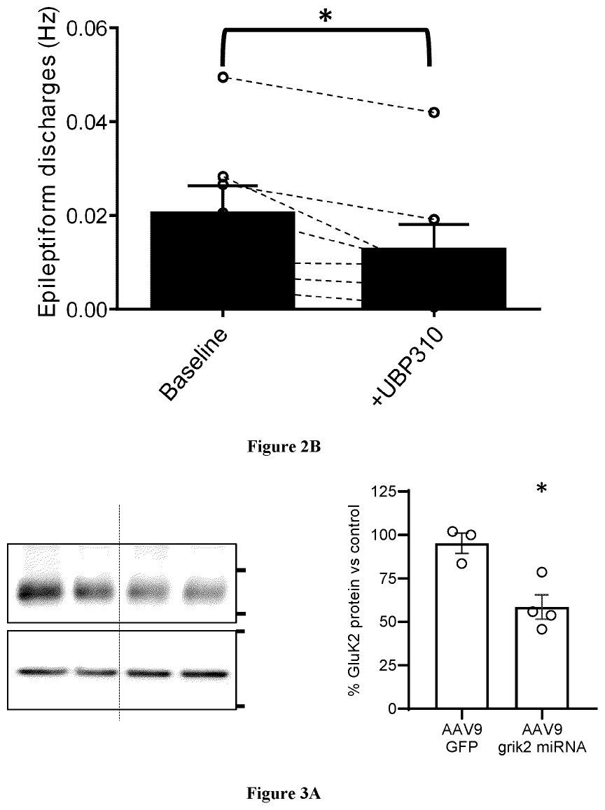

To investigate type II FCD, a rat model, induced by in utero electroporation of the recurrent pathogenic variant mTOR p.S2215F in the somatosensory cortex (see methods), was first utilized (Pelorosso et al., 2019). As expected, the electroporated somatosensory cortex demonstrated a typical cytoarchitectonic pattern of type II FCD (Pelorosso et al., 2019)(FIG. 1); this pattern included neuronal dyslamination and cytomegalic neurons highly-stained with antibody against phospho-S6 Ribosomal protein. Our data revealed that the blockade of GluK2-containing KARs, with 5 μM UBP 310 (Pinheiro et al., 2013), induced a significant reduction (by around 46%) in the frequency of spontaneous recurrent epileptiform discharges recorded in neocortical acute slices from rats with FCDII (FIG. 2A-B).

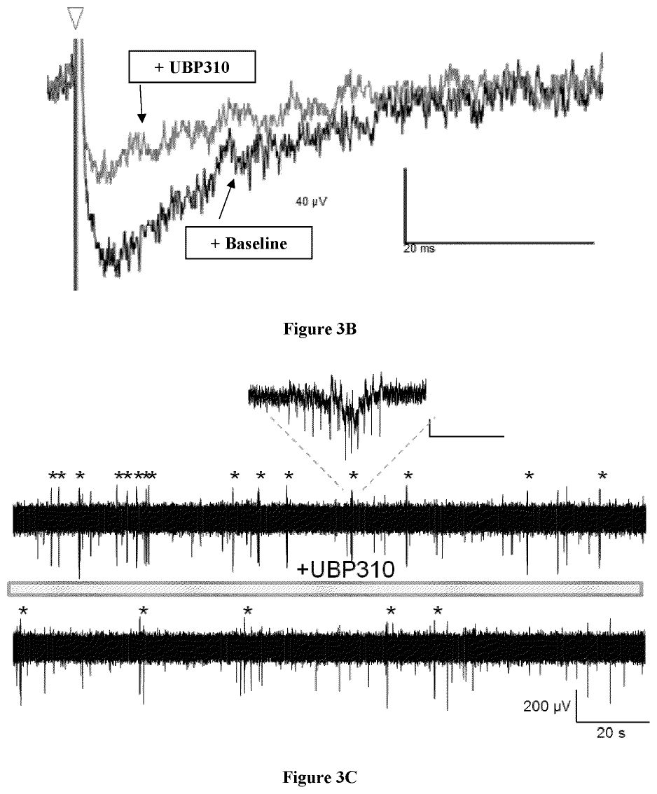

In a subsequent set of experiments, surgically resected cortical tissues were obtained from patients with FCD and drug-resistant epilepsy. Our findings revealed that: i) GluK2 receptors are highly expressed in cortical slices from a patient with intractable epilepsy; ii) GluK2 protein can be strongly reduced in the presence of AAV9 expression an antigrik2 miRNA (FIG. 3A). iii) The amplitude of evoked field EPSP (fEPSP) is strongly reduced in the presence of 5 μM UBP 310 (by approximately 50%, FIG. 3B), as well as the frequency of spontaneous recurrent epileptiform discharges (by around 70%, FIG. 3C), in acute cortical slices from a patient with FCD.

In conclusion, the results indicate that KARs play a significant role in cortical epilepsy circuits which are involved in FCD.

### EXAMPLE 2

### Material & Methods

Brains were freshly frozen in isopentane at −80 °C. Fresh frozen sections prepared with a Cryostat at 20 μm were air-dried and fixed by dipping in 4% paraformaldehyde in 0.1 M phosphate buffer (pH 7.2) for 15 min, before to be stock at −80 °C. For Immunohistochemistry, the sections were incubated with 10% normal donkey serum for 30 min at room temperature, followed by an incubation with a mixture of the following primary antibodies overnight at 4 °C with polyclonal rabbit anti-GluK2 antibody (dilution of 1:800, SYSY ref. 180003) or polyclonal rabbit anti-phospho-S6 ribosomal protein antibody (dilution of 1:1000, Cell signaling ref. 5364). The sections were then incubated with a mixture of Alexa Fluor 647 labeled species-specific secondary antibodies for 2 h at a dilution of 1:1000 (Thermo Fisher Scientific) and mount in Fluoromount+DAPI mounting medium. Images were taken with an epifluorescence microscope Leica DM5000 (Leica Microsystems, Nanterre, France).

### Results

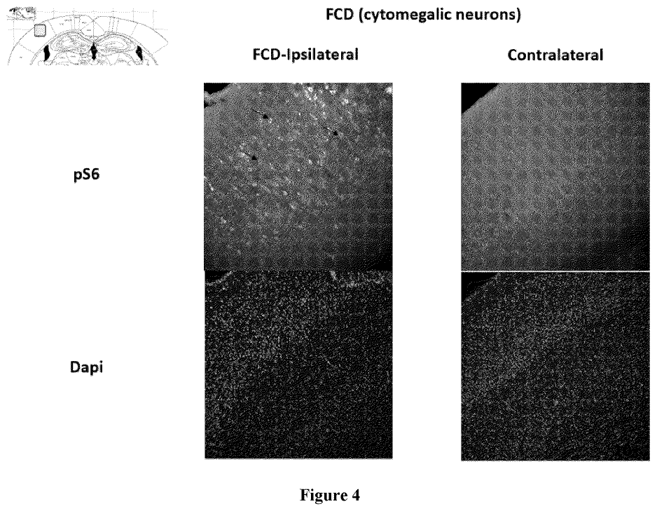

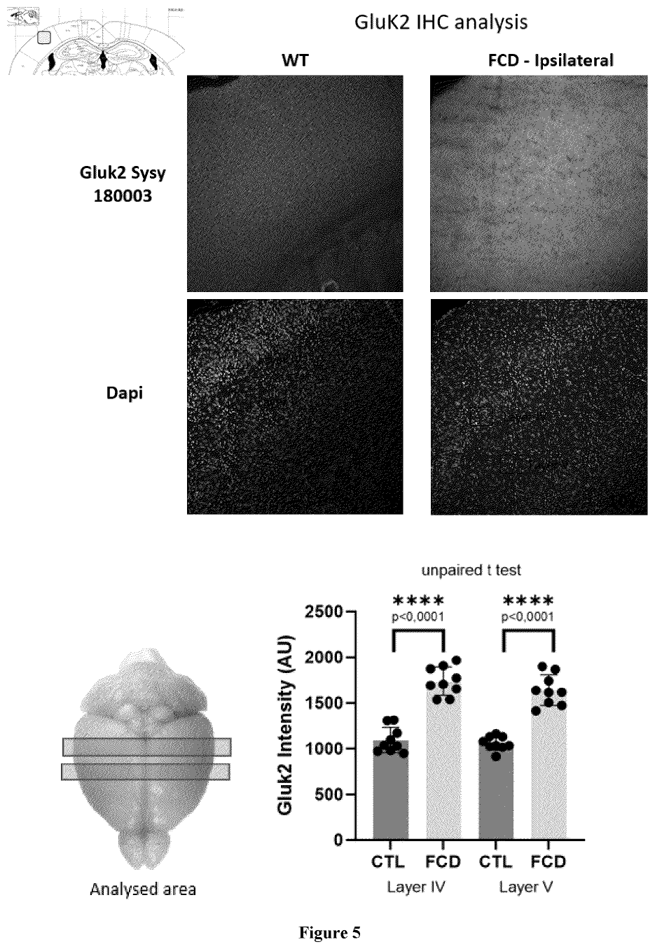

Immunostaining of pS6 in cytomegalic neurons in Focal Cortical Dysplasia is represented in FIG. 4. Immunostaining of GluK2 in Wild-Type and Focal Cortical Dysplasia Tissues is represented in FIG. 5. The graph illustrates the immunohistochemical (IHC) analysis of GluK2 expression in cortical layers IV and V, showing increased GluK2 expression in the FCD area compared to WT condition (control, CTL).

### REFERENCES

Throughout this application, various references describe the state of the art to which this invention pertains. The disclosures of these references are hereby incorporated by reference into the present disclosure.
Ackman J B, Aniksztejn L, Crepel V, Becq H, Pellegrino C, Cardoso C, Ben-Ari Y, Represa A (2009) Abnormal Network Activity in a Targeted Genetic Model of Human Double Cortex. Journal of Neuroscience 29: 313-327.
Baldassari S, Ribierre T, Marsan E, Adle-Biassette H, Ferrand-Sorbets S, Bulteau C, Dorison N, Fohlen M, Polivka M, Weckhuysen S, Dorfmüller G, Chipaux M, Baulac S (2019) Dissecting the genetic basis of focal cortical dysplasia: a large cohort study. Acta Neuropathol 138: 885-900.
Blümcke I et al. (2011) The clinicopathologic spectrum of focal cortical dysplasias: A consensus classification proposed by an ad hoc Task Force of the ILAE Diagnostic Methods Commission 1: The ILAE Classification System of FCD. Epilepsia 52: 158-174.
Englot D J, Chang E F (2014) Rates and predictors of seizure freedom in resective epilepsy surgery: an update. Neurosurg Rev 37: 389-405.
Lamberink H J et al. (2020) Seizure outcome and use of antiepileptic drugs after epilepsy surgery according to histopathological diagnosis: a retrospective multicentre cohort study. The Lancet Neurology 19: 748-757.
Le Duigou C, Savary E, Morin-Brureau M, Gomez-Dominguez D, Sobczyk A, Chali F, Milior G, Kraus L, Meier J C, Kullmann D M, Mathon B, de la Prida L M, Dorfmuller G, Pallud J, Eugéne E, Clemenceau S, Miles R (2018) Imaging pathological activities of human brain tissue in organotypic culture. J Neurosci Methods 298: 33-44.
Najm I, et al. The ILAE consensus classification of focal cortical dysplasia: An update proposed by an ad hoc task force of the ILAE diagnostic methods commission. Epilepsia. 2022 Aug.; 63(8): 1899-1919. doi: 10.1111/epi.17301. Epub 2022 Jun. 15.
Pelorosso C, Watrin F, Conti V, Buhler E, Gelot A, Yang X, Mei D, McEvoy-Venneri J, Manent J -B, Cetica V, Ball L L, Buccoliero A M, Vinck A, Barba C, Gleeson J G, Guerrini R, Represa A (2019) Somatic double-hit in MTOR and RPS6 in hemimegalencephaly with intractable epilepsy. Human Molecular Genetics 28: 3755-3765.
Pinheiro P S, Lanore F, Veran J, Artinian J, Blanchet C, Crépel V, Perrais D, Mulle C (2013) Selective Block of Postsynaptic Kainate Receptors Reveals Their Function at Hippocampal Mossy Fiber Synapses. Cerebral Cortex 23: 323-331.

Sisodiya S M, Fauser S, Cross J H, Thom M (2009) Focal cortical dysplasia type II: biological features and clinical perspectives. The Lancet Neurology 8: 830-843.
Ruiz, A., et al, 2005. Distinct subunits in heteromeric kainate receptors mediate ionotropic and metabotropic function at hippocampal mossy fiber synapses. 25, 11710-11718. doi: 10.1523/JNEUROSCI.4041-05.2005

1. A method of treating focal cortical dysplasia (FCD) in a subject in need thereof comprising administering to the subject a therapeutically effective among of an inhibitor of GluK2-containing kainate receptors (KAR).

2. The method according to claim 1, wherein the subject has an epilepsy.

3. The method according to claim 1, wherein the KAR is a GluK2/GluK5 receptor.

4. The method according to claim 1, wherein the inhibitor is selected from the group consisting of UBP310, UBP302, UBP316 and NBQX.

5. The method according to claim 1, wherein the inhibitor is an inhibitor of GluK2 subunit gene expression.

6. (canceled)

7. A therapeutic composition comprising an inhibitor of GluK2-containing kainate receptors for use in the treatment of focal cortical dysplasia (FCD) in a subject in need thereof.
